(19) United States
(12) Patent Application Publication
(10) Pub. No.: US 2003/0165917 A1
(43) Pub. Date: Sep. 4, 2003

(54) Isothermal amplification in nucleic acid analysis

(76) Inventors: Edwin F. Ullman, Atherton, CA (US); Ming Wu, Castro Valley, CA (US); Yen Ping Liu, Cupertino, CA (US)

Correspondence Address: HANA VERNY, PETERS, VERNY, JONES & SCHMITT LLP, SUITE 6, 385 SHERMAN AVENUE, PALO ALTO, CA, 94306, US

(21) Appl. No.: 10/219,195

(22) Filed: Aug. 14, 2002

Related U.S. Application Data
(60) Provisional application No. 60/312,505, filed on Aug. 14, 2001.

(51) Int. Cl. C12Q 1/68; C12P 19/34

(52) U.S. Cl. 435/6; 435/91.2

(57) ABSTRACT

A method for detection of nucleic acid targets using as reagents: (1) a stem loop probe having a long strand for hybridizing to a target, a short strand, usually with a free 3′-end for extension, hybridized to a portion of the long strand and a linker forming a loop and joining the short and long strands; and (2) a hybridizing reagent that hybridizes to the short strand when released by the target. The target is detected by extension of one or both the probe or hybridizing reagent along each other. A circular hybridizing reagent can be employed with DNA polymerase for concatenated extension of the probe 3′-end or extension of the 3′-end of the probe along a hybridizing reagent having a promoter sequence for forming transcripts of at least a portion of the long strand or the hybridizing reagent. A non-cleavable restriction site consensus sequence in the linker, where the hybridizing reagent is extended with dNTPs and DNA polymerase and the extended sequence is cleaved with a restriction enzyme, so that chains may be repetitively produced and cleaved. The presence of the target nucleic acid is established by detecting the concatenated chain, the RNA transcripts or the repetitively produced chains.

This application is a continuation in part of provisional application serial No. 60/312,505, filed Aug. 14, 2001, the entire contents of which is incorporated herein by reference.

### BACKGROUND OF THE INVENTION

1. Field of the Invention

The field of this invention is the detection of nucleic acids in a sample.

### BACKGROUND AND RELATED DISCLOSURES

The response of cells to changes in the environment or their status is to change the protein profile in the cell, modify existing compounds, particularly proteins, in the cell, transport of proteins to different sites in the cell, secrete compounds, formation of complexes, and the like. The processes involve metabolism and catabolism, with degradation of many proteins and up and down regulation of the expression of many proteins. The first stage in the expression of a protein is transcription to an mRNA, followed by translation of the mRNA to a protein by a ribosome. While the protein may be subject to further modification to be active, as a first iteration of the status of the cell, determining the presence and amount of the mRNA is of interest.

Determining the amount of a single mRNA or a profile of mRNA's as indicative of the effect of a change of environment or status of a cell provides for insight into the response of the cell to the environmental changes. Environmental changes can vary from infectious diseases, inflammatory responses, responses to autoimmune attack, response to toxic or therapeutic agents, and the like. By measuring the change in the mRNA profile, one can usually obtain an indication as to how the cell is responding and the nature of the response. One can track the response over time to evaluate the cellular ability to deal with the change in the environment and obtain an indication of the role different proteins play in this response. Also of interest is change of status, such as a normal cell becoming neoplastic or metastatic. For both diagnostic and therapeutic purposes it is of interest to know what proteins are involved in establishing the status and which may serve as targets or as a diagnostic. There is also an interest in changes in the mRNA profile in relation to the development of drugs. One is not only interested in knowing that the drug is successful in binding to a target, but the consequences of such binding, as well as the side effects resulting from binding of the drug to other entities present in the cell are also of interest.

Because of the manifold purposes associated with determining the presence of one or more mRNAs many different protocols have been developed. Depending on the purpose for the determination, one may be interested in the speed of the assay, the number of reagents and stages, ease of manipulation, amount of sample required, equipment required, accuracy of the result, sensitivity and the like. Methodologies that provide greater numbers of the desired characteristics remain of interest today.

Use of a probe having a complementary sequence forming a stem and complementary to a target sequence is used, where one arm of the stem has a fluorescer and the other arm has a quencher. U.S. Pat. Nos. 5,118,801; 5,312,728; 5,925,517 and 6,103,476. Use of probes in conjunction with promoter sequences for producing and identifying transcription products is described in U.S. Pat. Nos. 5,169,766; 5,629,153; and 6,025,133 and WO89/06700 and 90/120,652. Use of a circular template is described in U.S. Pat. No. 6,054,274. Use of three way junctions are described in U.S. Pat. No. 5,857,430 and Wharam, et al., Nucleic Acids Research, 29:54 (2001). A description of T7 RNA polymerase is found in Cazenave and Uhlenbeck, Proc. Natl. Acad. Sci. USA, 91: 6972-6976 (1994).

All publications and patent applications mentioned in this specification are herein incorporated by reference to the same extent as if each individual publication or patent application was specifically and individually indicated to be incorporated by reference.

### SUMMARY OF THE INVENTION

Methods and compositions are provided for determining at least one target nucleic acid in a mixture of nucleic acids employing a probe, a hybridizing reagent, and one or more phosphate bond-forming enzymes associated with any required nucleotide triphosphates to form a nucleic acid chain. The probe comprises an oligonucleotide having a stem/loop structure and an overhang, where the long strand can be hybridized to the target nucleic acid and the short strand is hybridized to a portion of the long strand. The hybridizing reagent will be an oligonucleotide capable of hybridizing at the short strand end of the stem/loop probe and to at least a portion of the short strand. Various techniques are employed for detecting the target by modifying the 3′-end of the probe or hybridizing reagent when the probe is hybridized to the target. These methods usually involve amplification, such as including the use of a promoter in conjunction with a RNA polymerase, a restriction site where only one strand is cleaved and is then displaced by extension with a DNA polymerase, or a circular hybridizing reagent, where concatenated repeats are produced. Detection of the amplified nucleic acid may take many forms. An exemplary one is the use of labeled nucleotides that become incorporated in the amplified strands, which may then be detected as indicative of the presence of the target nucleic acid. The process is isothermal, and allows for amplification in a single stage or sequential stages in a single vessel, where all of the reagents are compatible.

### BRIEF DESCRIPTION OF THE FIGURES

FIG. 1 is a schematic representation of a protocol used for detection of RNA and RNA mixtures using a fluorescent RNA which binds to stem/loop probe on the surface.

FIG. 2 is a schematic representing a protocol used for detection of RNA or RNA mixtures using a fluorescent RNA which binds RNA target transcripts to template probe on the surface.

FIG. 3 is a schematic representation of a variation of the method utilizing a ligase and ATP instead of DNA polymerase (Klenow).

FIG. 4 is a schematic representation of a variation of the method where the target hybridizes with the stem/loop probe causing the short strand not to be hybridized.

FIG. 5 is a schematic representation of a variation of the method wherein the target nucleotide binds to a stem/loop probe with the short strand of the 3′ end. Upon release of the short strand from hybridization, the short strand is available to bind to circular DNA.

FIG. 6 is a graph illustrating sensitivity of detection signal of Bac-PR 54 and Bac-PR 44 at variable concentrations of target nucleotide.

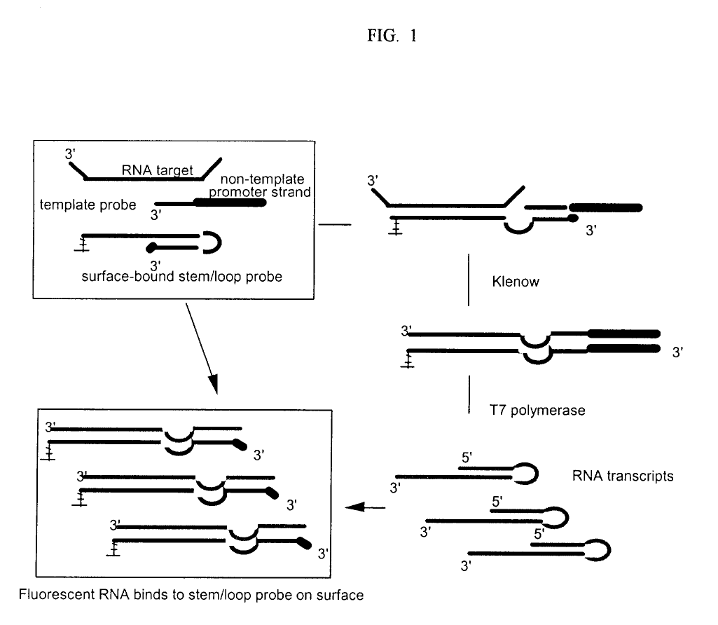

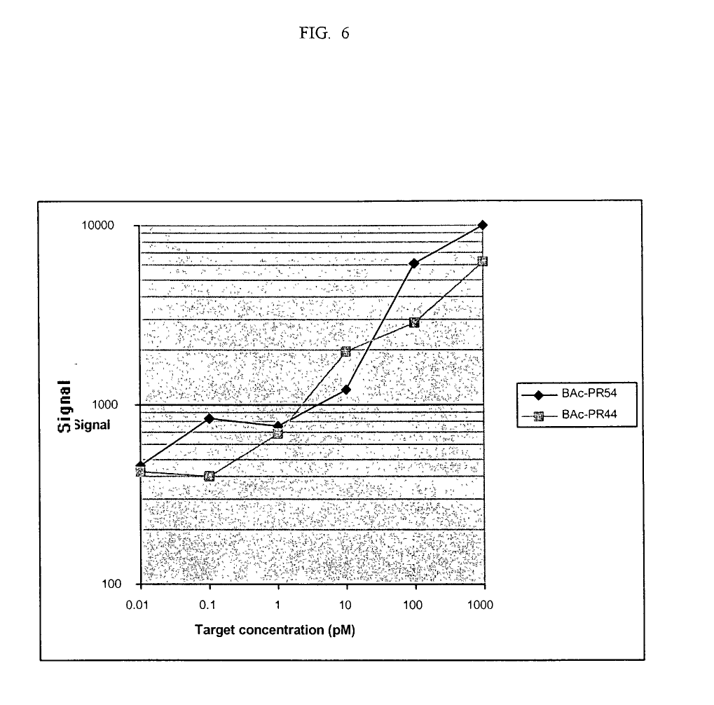

FIGS. 1-5 are cartoons of different embodiments of the processes of the subject invention.

### DETAILED DESCRIPTION OF THE INVENTION

Methods and compositions are provided for detection of at least one target nucleic acid, usually a mRNA, DNA or cDNA, in a mixture of nucleic acids. The methods are isothermal and allow for amplification in a single vessel and in a single or sequential stage. The methods employ at least two nucleic acid containing reagents: a stem/loop probe; and a hybridizing reagent that is usually substantially complementary to at least the short strand end, usually the 3′-end, of the stem/loop probe. That is, the short strand may have a 3′- or 5′-end. In addition, there will usually be one or more, occasionally at least two phosphate bond forming reagents capable of linking a 3′-end to a nucleotide, usually including phosphate-bond forming enzymes, particularly including nucleic acid polymerases, and the necessary (d) NTPs or a ligase. In carrying out the method, the components of the determination are combined and the mixture incubated for sufficient time for binding of target nucleic acid to the stem/loop probe and modification of the 3′-end of the stem/loop probe, which usually involves amplification of a nucleic acid product. The product may then be detected in a variety of ways as indicative of the presence, and desirably, the amount of target nucleic acid in the mixture. Conveniently, the product may be sequestered for analysis.

Generally, the method employs as reagents a stem/loop probe having a long strand hybridizing to at least a portion of a target nucleic acid, a short strand at the 3′-end of the probe hybridized to a portion of the long strand, although the short strand may be at the 5′-end of the probe, and a linker to the long and short strands as a loop, and a hybridizing reagent which has a hybridizing region to at least the -end of the short strand. By “end” is intended a sequence of nucleotides, usually at least about 5 nucleotides to provide for specific hybridization under the conditions of the assay. The method is performed by bringing together or contacting with each other the sample, the stem/loop probe, and the hybridizing reagent, wherein the hybridizing reagent hybridizes with the short strand when said target nucleic acid is present in said sample and binds to the probe; followed by extending one of the 3′-end of the short strand hybridized or the hybridizing reagent, when the short strand and the hybridizing reagent are hybridized together; and detecting the extension as indicating the presence of the target nucleic acid. Preferred methods of extension will be as a result of ligating an oligonucleotide complementary to a portion of the hybridizing reagent, usually forming an active promoter or using DNA polymerase extension with a DNA polymerase and dNTPs, usually to create an active promoter. This means that the 3′-end is capable of reacting with a triphosphate to form a phosphate bridge.

In carrying out the method, the stem/loop probe is contacted with the sample comprising a complex mixture of nucleic acids and the hybridizing reagent, the enzyme(s) appropriate for the protocol and, when needed, the appropriate nucleotide triphosphates for nucleic acid formation, and any other reagents that are appropriate under isothermal conditions for bond formation. While the protocol may be carried out in stages, the method allows for the combination of all of the reagents at the same time, so that different reactions may proceed simultaneously to provide the amplified product for analysis. In one embodiment, the stem/loop probe and sample are combined together under hybridizing conditions where target nucleic acid can bind to the stem/loop probe and the stem is opened to provide the short strand as a single strand. Desirably, any portion of the sample that is not bound to the stem/loop probe is washed away. The stem/loop probe will have means for sequestering the complex of the stem/loop probe and the target nucleic acid to allow for removal of the remaining portion of the sample. The hybridizing reagent is then added under hybridizing conditions, where the hybridizing reagent binds to the single stranded short strand. Amplification is then initiated. Each of the methodologies requires the opening of the hairpin or stem/loop by binding of the stem/loop probe to the target nucleic acid. The release of the short strand from binding to the long strand permits the succeeding binding of the hybridization reagent followed by amplification. There are different modes of amplification that are described below.

The protocols may be divided into different subgenera. The first protocol to be generally described employs a promoter that can be obtained by DNA polymerase or ligase extension, where one strand of a promoter present in the hybridizing reagent is made or is double stranded and can support transcription. The double stranded promoter is formed by DNA polymerase extension of or ligation to the 3′-end of the short strand of the stem/loop probe of one strand, usually the template strand of the promoter, while the complementary strand, usually the non-template strand of the promoter is joined to the hybridizing region of the hybridizing reagent. When the stem/loop probe has a 5′ end at the short strand, the hybridizing reagent will have two strands, a first strand complementary to the short strand and a shorter strand hybridized to the longer strand to form a promoter that is contiguous to the 5′ end of the stem/loop probe and can be ligated to the stem/loop probe. By having the double stranded portion of the hybridizing probe defining a promoter, RNA copies of the long strand or the short strand can be obtained. In this instance as in any protocol where the hybridizing reagent is duplexed, the hybridizing reagent may form a second stem/loop probe, with a linker that is innocuous or may be used further for identification, sequestering, etc. The presence of an RNA polymerase results in transcription of repetitive copies of RNA.

A second protocol employs the loop of the stem/loop probe having a restriction enzyme sequence, where at least one of the nucleotides is modified to prevent cleavage of the strand. By extending the hybridizing reagent bound to the short strand with DNA polymerase, a sequence will be formed that includes the consensus sequence for the restriction enzyme and a sequence complementary to the long strand of the stem/loop probe that comprises the target nucleic acid sequence. A restriction enzyme cleaves the extended sequence at the restriction site and the portion of the extended sequence complementary to the long strand of the stem/loop probe is displaced by extension of the cleaved 3′-end by DNA polymerase. The newly extended sequence is again cleaved and the process proceeds repetitively.

A third protocol employs a circular nucleic acid that includes the sequence hybridizable to at least a portion of the short strand as the hybridizing reagent. In the presence of DNA polymerase, when the circular hybridizing reagent binds to the short strand, a concatenated repetitive strand complementary to the circular hybridizing reagent is obtained.

The method finds use in any situation where there is an interest in detecting one or more target nucleic acids, particularly where multiplexing is desirable. The samples may be from any source. The sample that serves as the source of the nucleic acid to be amplified and analyzed may come from viral nucleic acid, prokaryotic or eukaryotic nucleic acid, unicellular organisms, e.g. bacteria and protista, invertebrates, vertebrates, particularly mammals, etc. The subject methodology is particularly applicable to complex mixtures having large numbers of different nucleic acids, where the target nucleic acid may be a single target or a plurality of targets, both DNA and RNA, particularly mRNA. The sample will provide at least about 1 attomole of each of the target nucleic acids, usually at least 1 femtomole and frequently at least one picomole, but may include as low as a single copy, particularly when analyzing single cells. Obviously, much larger amounts of target nucleic acid may be used.

Depending on the source of the sample, the sample may be subjected to various prior processing before being used in the transcriptional amplification. The source may be individual cells of the same type or mixed type, as in tissue, biopsy, swab, blood, lymph fluid, CNS fluid, urine, saliva, waste water, soil, effluents, drinking water, cooling water, foods, agricultural products, drugs, etc., may be a single culture, cell line, primary cells, or the like. The cells may have been subject to prior separation by means of FACS, immunoseparation using antibodies that bind to specific markers, or other selection means. Depending on the nature of the sample, the sample may be subject to concentration, precipitation, filtration, particularly microfiltration, chromatography, etc. For cells, the cells will be lysed by any convenient means, using detergents, mechanical disruption, e.g. sonic disruption, etc. Where RNA is the target, RNase inhibitors, such as RNA Guard® may be added, and the sample otherwise treated to prevent degradation of the RNA. Nucleic acid precipitation may be employed to isolate the DNA, which may then be degraded using restriction enzymes, mechanical disruption, etc., and rendered single stranded by heating, treatment under alkaline conditions, exposure to low ionic strength media, etc. Nucleic acid preparation can follow well recognized techniques, such as those described in “Molecular Cloning: A Laboratory Manual” (Cold Springs Harbor Laboratory Press, Cold Spring Harbor, N.Y., 1989), Mach et al., The Annual of Biological Chemistry, 261:11697-11703 (1986); Jeffries et al., J. of Biol. Chem., 269:4367-4372 (1994); and U.S. Pat. Nos. 5,654,179 and 5,993,634.

The individual reagents will now be considered. The reagents may vary to some degree with different protocols, but there will be common features to each of the reagents.

The first reagent to be considered will be the stem/loop probe. The stem/loop probe will normally be DNA, although in some instances portions of the stem/loop probe may be PNA or RNA, when the RNA provides an advantage or may be substituted for the DNA without a detrimental effect on operability. This will become clearer as the individual protocols are discussed in detail. The stem/loop probe may be divided into three segments, a long strand hybridizable to a target nucleic acid, a short strand comprising the 3′- or 5′-end of the probe, usually the 3′-end, comprising at least a portion hybridizable to a portion of the long strand, which defines the hybridizable region, and a linker or loop joining the two strands at their respective ends. There may be one or more (1-3) non-hybridizing nucleotides at the -end of the short strand that do not hybridize to the long strand. The non-hybridizing nucleotides at the terminus of the short strand are provided in order to avoid DNA polymerase catalyzed extension of the short strand along the long strand. Where the extension is not of concern, the non-hybridizing terminal nucleotides need not be present.

The linker or loop strand covalently joins the long and short strands. In some protocols it will be desirable for the linker to be a chain of atoms that includes other than one or more nucleotides or no nucleotides. Usually however, the linker will be at least one nucleotide, usually at least about 3 nucleotides and generally in the range of about 3 to 30, usually about 3 to 20 nt. Since there will usually be no advantage in having a very long linker, for purposes of economy, the linker will be kept as short as the protocol allows. Desirably, at the juncture of the linker with the short and long strands, nucleotides are provided that are complementary, so as to hybridize. Usually, the number of nucleotides involved resulting in the lengthening of the stem portion of the stem/loop probe will be in the range of from 1 to 4, more usually 1 to 3 nucleotides. Extending the stem portion with nucleotides that are not involved with the target nucleic acid serves to inhibit adventitious binding of the hybridizable reagent in the absence of bound target.

The long strand has no operable limit, but for convenience it will generally be in the range of about 15 to 50 nt, usually from about 15 to 35 nt and at least about 5 nt greater than the short strand hybridizable region. The short strand will be at least about 5 nt, usually at least about 8 nt and not more than about 20 nt in the hybridized region.

In the protocols employing DNA polymerase, the 3′ end of the stem/loop probe, usually the 3′ end of the short strand, will usually be inhibited from being extended along the long strand as the template, but may not be inhibited from extension along the hybridizing probe. This can be achieved in a variety of ways, depending upon the manner in which the DNA polymerase is employed in the protocol. Most conveniently, the 3′ end may have at least one nucleotide that is not complementary to the long strand, so as to be single stranded. Usually there will be not more than 3 mismatches, more usually not more than about 2 mismatched nucleotides. Alternatively, for some protocols, an unnatural base, such as a PNA base may be present at a position in the long strand that is immediately downstream from the portion hybridized with the short strand, so as to preclude the long strand from serving as a template for the polymerase.

The total number of nucleotides in the stem/loop probe will usually be at least about 25 nt and not more than about 100 nt, generally in the range of about 40 to 75 nt.

The loop may serve a number of functions, depending on the protocol employed. The loop may have one or more modified nucleotides, e.g. phosphorothioates, in a restriction enzyme consensus sequence, which results in the strand being resistant to cleavage by a restriction enzyme, while allowing for cleavage of the complementary strand. For restriction enzymes and consensus sequences, see, for example, Eckstein, Biochem. Soc. Trans., 14:204-5 (1986). In another protocol, the loop sequence may serve to provide a convenient identification sequence for the target sequence. As indicated above, the ends of the loop may serve to extend the stem beyond the target associated stem portion.

The stem/loop probe may be in soluble or insoluble form, that is, bound to a support. The support may be particles, including magnetic particles, latex particles, dextran particles, etc., where the nature of the particles do not interfere with the determination, and where the particles may serve to segregate the stem/loop probe bound to the amplified product, to a surface of a vessel or plate, particularly as an array, or the like. Usually the 5′ end of the long strand or a group comprising the linker will serve as the site for linking to the surface, although other sites of attachment can be used. Various linking groups may be employed that are extensively described in the literature, for linking during synthesis of oligonucleotides or other convenient linkers. The linkers will be selected to be inert under the conditions of the determination and will minimize steric interference with binding to the stem/loop probe. For a description of linking groups, see, for example, Pease, et al., Proc. Natl. Acad. Sci., 91:5022-5026 (1994); Khrapko, et al., Mol. Biol., (Mosk USSR), 25:718-730 (1991); Simpson, et al., Proc. Natl. Acad. Sci. USA, 92:6379-6383 (1995); and Guo, et al., Nucleic Acids Res., 22:5456-5465 (1994).

The hybridizing reagent will have a region hybridizable to the short strand (the “hybridizing region”), and optionally one or more, usually not more than 3, bases of the loop. Preferably, the hybridizing reagent will be designed to hybridize with the 3′-end of the stem/loop probe when it is hybridized to the stem/loop probe or the 3′-end of the hybridizing reagent will be part of the hybridizing region.

The hybridizing region may be the entire hybridizing reagent or be bound to various other sequences. In some instances, the 5′-end of the hybridizing region will be joined to the 3′-end of one strand of a promoter sequence, preferably the non-template strand of a promoter sequence, which when bound to its complementary strand provides a “holopromoter.” By holopromoter is intended that a functional double stranded promoter is present. Additionally, an arbitrary sequence that may serve for sequestering the amplified product may be present between the hybridizing region and the promoter sequence, when the promoter sequence is a non-template strand, at the 5′-end of the promoter sequence, when the promoter sequence is a template strand. The two strands of the promoter may be joined by a linking group to form a stem/loop structure. In another embodiment, the arbitrary sequence serves to provide a circular template to produce a concatenated product.

Because of the variety of hybridizing reagents, the hybridizing reagent will usually have a minimum number of 5 nt, usually at least about 10 nt, but a maximum number will be arbitrary. For the most part, the hybridizing reagent will usually be fewer than 100 nt in a single strand, usually fewer than 75 nt, where in many instances the length of the hybridizing reagent will be a matter of economics and convenience. Of course, the circular hybridizing reagent may be much larger, being 200 nt or greater, but this will usually be unnecessary.

In some instances, where the hybridizing reagent comprises the non-template strand of the promoter an arbitrary sequence that can serve as an identifying sequence can be present between the hybridizing region and the non-template strand. A probe comprising a sequence hybridizing to the arbitrary sequence may then be provided that will bind to and thereby facilitate detection of transcripts.

Instead of having the stem/loop probe bound to a surface, the hybridizing reagent may be bound to the surface. The same considerations for binding of the stem/loop probe to the surface will be applicable to the hybridizing reagent. Either excess stem/loop probe or hybridizing reagent may serve to capture amplified product, so that the amplified product may be sequestered by one of the bound reagents.

In some protocols, instead of having transcripts of at least portions of the stem/loop probe, one may produce transcripts of at least portions of the hybridizing reagent. The hybridizing reagent would then comprise beginning at its 3′-end, optionally a blocking entity for preventing extension of the hybridizing reagent, the hybridizing region, the template strand of the promoter, and the arbitrary sequence. Such a hybridizing reagent will provide transcripts complementary to the arbitrary sequence that can hybridize to the hybridizing reagent that is bound to a surface to facilitate detection. The arbitrary sequence may serve a number of different functions, such as identifying the target sequence, a sequence for binding to a sequestering sequence, or other function for identifying and quantifying the target sequence.

The hybridizing reagent may also comprise both strands of a promoter that are attached, conveniently by a loop. In this case the hybridizing region comprising the 3′-end of the hybridizing reagent is linked to the 3′-end of the non-template strand of a promoter. The non-template strand is joined at its 5′-end through a linker to the template strand of the promoter to form a hairpin (stem and loop) hybridizing reagent that terminates in a 5′-phosphate. Upon binding a target nucleic acid to the stem/loop probe, the 3′-end of the stem/loop probe hybridizes to the hybridizing region at a site contiguous to the bound template strand. A DNA ligase then ligates the stem/loop probe to the hybridization reagent, which enables RNA polymerase to initiate transcription along the stem/loop probe.

A ligating reagent is particularly useful when the target nucleic acid is DNA and a DNA polymerase is employed in the protocol. Under these conditions the hybridizing reagent will often be able to hybridize with and extend upon the target DNA and render it unavailable for binding to the stem/loop probe. This problem is minimized by using a hybridizing reagent that has a very short hybridizing sequence, usually 2 to 5 bases. However, the short hybridizing sequence will prevent extension of the hybridizing reagent along the stem/loop probe. This problem is circumvented by extending the 3′-end of the short strand of the stem/loop probe with a ligating reagent that is able to bind to the hybridizing reagent. Once ligation has occurred a longer portion of the hybridizing reagent can bind to the elongated short strand and be extended by DNA polymerase.

The enzymes that are used are enzymes that can be used to form phosphate bonds, normally involving a free 3′-hydroxyl group and a nucleotide triphosphate or enzymes that cleave a phosphate bond. Such enzymes include DNA polymerases, RNA polymerases, ligases and restriction enzymes. For some protocols, the DNA polymerases that are employed will be free of editing capability, such as the Klenow fragment, which is commercially available and can be effectively used. Examples of other DNA polymerases include P. polycephalum á or â, Taq polymerase, Sequenase, bacteriophage 32, T3, T7, SP6, and reverse transcriptases. For the RNA polymerase promoters and their concomitant polymerases, a T promoter, and particularly the T7 promoter, although other T promoters, such as T3 can also find use, as well as other bacteriophage promoters, such as SP6 and BA11.

The T7 promoter has conserved residues from −17 to +6 relative to the start site of transcription, where the promoter may be considered to be divided into two domains, an initiation domain from −4 to +5 and a binding domain from −5 to −17. The initiation domain can be substantially eliminated, so that the nucleotides from −1 to −17 are all that are required. Single base changes in the binding domain of the T7 promoter reduce or eliminate promoter binding, but have little effect on the initiation of transcription. By way of contrast, single base changes in the initiation domain of the promoter have little effect on promoter binding but reduce the rate of initiation. The base pairs at −9, −10 and −11 appear to distinguish between T7 and T3, while the base pairs at −9 and −8 distinguish between T7 and SP6. In addition, nucleotides from the 5′ and 3′ ends may be removed while still retaining transcription initiation activity. Since any change tends to reduce the transcription rate, these modifications are generally not desirable.

The promoter region for bacteriophage RNA polymerases will usually be at least about 17 bp, will usually have at least about 90% of the base pairs conserved, usually at least about 95% and more usually 100% conserved of the naturally occurring promoter region. Usually chain extension of the stem/loop will produce the complementary sequence to a single stranded promoter sequence of the hybridizing reagent. However, the second strand of the promoter sequence may also be introduced by ligation of a promoter sequence to the 3′-end of the stem/loop probe with the hybridizing reagent acting as a template. In this case, the two strands of the holopromoter will usually have not more than 3, usually not more than 2, bases of the promoter region mismatched. For the most part, only the promoter region of −1 to −17 will be used and even in this region substantial variation is permitted while still retaining a substantial portion of the maximal activity.

Any commercially available ligase, polymerase or restriction enzyme having the appropriate characteristics can be used and the conditions employed will be those recommended by the supplier.

For the most part, the protocols will be carried out at temperatures in the range of about 15 to 50° C., more usually in the range of about 25 to 45° C. The temperature chosen for the determination will be related to optimize the activity of the enzymes employed in the protocol. Various aqueous solutions may be used for the determination, particularly buffered solutions compatible with any enzymes employed. Buffers that can find use include phosphate, Tris, borate, MOPS, bicarbonate, etc. The pH will generally be in the range of about 6 to 8, selecting a buffer, concentration, ionic strength and pH that provides at least substantially optimum activity for the enzyme(s) employed. Buffer concentrations will generally be in the range of about 20 to 200 mM, where conventional auxiliary agents will be included, such as salts, polyamines, minor groove binders, intercalating agents, proteins, antioxidants, chelating agents and the like. Salt concentrations will normally range from 0.01 to 5M, more frequently from 0.1 to 2M. The concentrations of the soluble oligonucleotide reagents, such as the stem/loop probe and the hybridizing reagent, will generally be in the range of about 0.01 to 2×10³ nM, more usually in the range of about 0.1 to 2×10³ nM. Any nucleotide triphosphates that are required will generally be at a concentration in the range of about 0.1 to 10 mM. Usually the concentration of the polymerases will be at about 0.5 to 5 units for the DNA polymerase and about 5 to 25 units for the RNA polymerase in a volume of from about 10 to 50 il. The other enzymes will be used at conventional concentrations as indicated by their suppliers.

A general aspect of a number of the protocols is the creation of an active promoter for transcribing at least a portion of the stem/loop probe and/or another sequence as transcription products. The active promoter, which will be dsDNA, can be formed by extending with DNA polymerase the 3′-end of the stem/loop probe to make a strand complementary to the promoter sequence. Usually the complementary strand comprises the promoter template strand and the promoter sequence in the hybridizing reagent is a non-template strand at the 5′-end of the hybridizing region. Alternatively, the hybridizing reagent comprises the template strand of a promoter 5′ of the sequence hybridizing to the short strand of the stem/loop probe and an arbitrary region to serve as a transcription template that is 5′ of the template strand. In this case the complementary strand arising from chain extension is the non-template strand of the promoter. Therefore, depending on the manner in which the different reagents are devised, one may produce a transcript of some or the entire stem/loop probe or a transcript of a portion of the hybridizing reagent. These embodiments share the requirement that an active promoter for transcription of at least a portion of the hybridizing reagent or the stem/loop probe is not present until an enzymatic reaction has occurred, either DNA polymerase copying of the single stranded promoter sequence or ligation of the hybridization reagent to the stem/loop probe having the template strand of a promoter sequence for the ligation.

The other embodiments provide amplification without use of a promoter. In one embodiment amplification is achieved by having a restriction enzyme consensus sequence in the loop, where one or more of the nucleotides have been modified to prevent cleavage of the loop strand, while allowing for cleavage of the complementary strand. See, Eckstein, supra. The determination is performed in the presence of DNA polymerase, dNTPs, and the restriction enzyme, so that each time the strand is extended from the restriction site, the strand is cleaved and a new strand is formed displacing the prior strand. In this way an amplified DNA product of the target sequence is obtained.

A DNA product is also obtained by having a circular hybridizing reagent. Extension of the short strand at its 3′ end along the circular hybridizing reagent with DNA polymerase and dNTPs provides a concatenated continuous strand of copies of the circular hybridizing reagent, where the long continuous strand will include many copies of the portion of the short strand to which the hybridizing reagent bound.

Detection of the amplified sequences may take many forms. Conveniently, one may use labeled nucleotide triphosphates for incorporation into the amplified product. The labels may be directly detectable, such as fluorescent and chemiluminescent labels, electrochemical labels, and the like, or indirectly detectable, such as ligands that bind to labeled receptors, such as biotin-streptavidin, digoxin-antidigoxin, etc. where the proteins are labeled with the directly detectable labels. The amplified products may be used as linkers between bound complementary sequences and labeled sequences. In multiplexing, arrays can be used of the different target sequences, so that each of the amplified sequences will be bound at the specific site where the complementary sequence is bound. The amplified product may then serve to bind a labeled complementary sequence at each site, or labeled antibodies to RNA/DNA hybrids, or other conventional technique may be employed for detection. The amplified products may be separated using capillary electrophoresis, HPLC, or the like, and detected by various techniques described above, particularly incorporation of labeled nucleotides.

A variety of fluorescent labels are available, such as fluorescein, rhodamine, Texas red, porphyrins, phthalocyanines, umbelliferone, etc. The choice of the label will, for the most part, be arbitrary, so long as the desired sensitivity is achieved.

To further illustrate the subject invention, a number of exemplary protocols will be described, as shown in the figures. The first example is shown in FIG. 1. This example is best suited to detection of RNA and mixtures of RNA such as mRNA measurements required for cell profiling by expression analysis. The stem/loop probe is a DNA sequence. The long strand of the stem/loop probe can hybridize to target nucleic acid. The short strand is initially hybridized to the long strand except for its 3′ end, which comprises a 1-3 base sequence that does not hybridize to the long strand. The purpose of this non-hybridized segment is to prevent chain extension of the short strand along the long strand catalyzed by a DNA polymerase. Upon binding to the target, the short strand is displaced and becomes available for binding to the hybridizing reagent, which is complementary to the 3′ end of the short strand and comprises the non-template strand of a T7 promoter. The 3′ ends of the hybridizing reagent and the short strand are then extended by a DNA polymerase and dNTPs including optionally a detectably labeled dNTP, to provide a fully double stranded DNA comprising a T7 promoter. T7 polymerase and rNTPs then result in the formation of multiple RNA transcripts, which are complementary with and bind to the stem/loop probe. Detection of the transcripts can be by incorporating a detectable label during transcription and binding the transcripts to sites on a surface. Suitable binding agents that may be attached to the surface are the stem/loop probe and sequences complementary or antibodies to DNA/RNA heteroduplexes. By placing different stem loop probes in an array the method permits multiple targets to be assayed simultaneously. When the stem/loop probe is attached to a surface, in a predetermined array, labeled antibodies to RNA/DNA heteroduplexes are particularly useful for detecting the formation of the specific heteroduplex without the need for transcripts to be labeled.

There are several modifications of this process that are substantially equivalent. For example the hybridizing reagent may have an arbitrary sequence present between the 3′ end sequence that is complementary to the stem/loop probe, i.e. the hybridizing region, and the non-template promoter strand. The unhybridized 1-3 base 3′ end of the stem/loop probe is designed to hybridize to this arbitrary sequence to permit chain extension along the hybridizing reagent. An arbitrary sequence may also be present at the 5′ end of the hybridizing reagent. Preferably however the short strand of the stem/loop probe other than its 1-3 base unhybridized 3′ end will differ in sequence from the target sequence to which it becomes bound by fewer than two nucleotides.

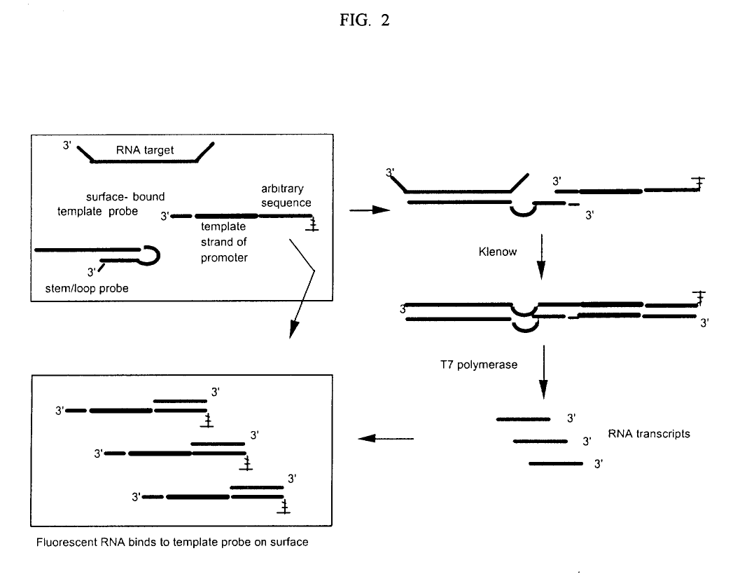

When the arbitrary sequence is at the 5′-end of the hybridizing reagent, the promoter sequence of the hybridizing reagent will usually be the template strand as shown in FIG. 2. The hybridizing reagent may be immobilized on a surface and the stem/loop probe will usually be in solution. Extension of the stem/loop probe along the hybridizing reagent leads to formation of a holopromoter capable of initiating transcription of the arbitrary sequence. Multiplexed analysis of different targets can be achieved by using a different hybridizing reagent for each target that has an arbitrary sequence that codes for the specific target or a group of target nucleic acids. The presence of a specific target can then be determined by the binding of arbitrary sequence transcripts to different sites in an array of the hybridizing reagents.

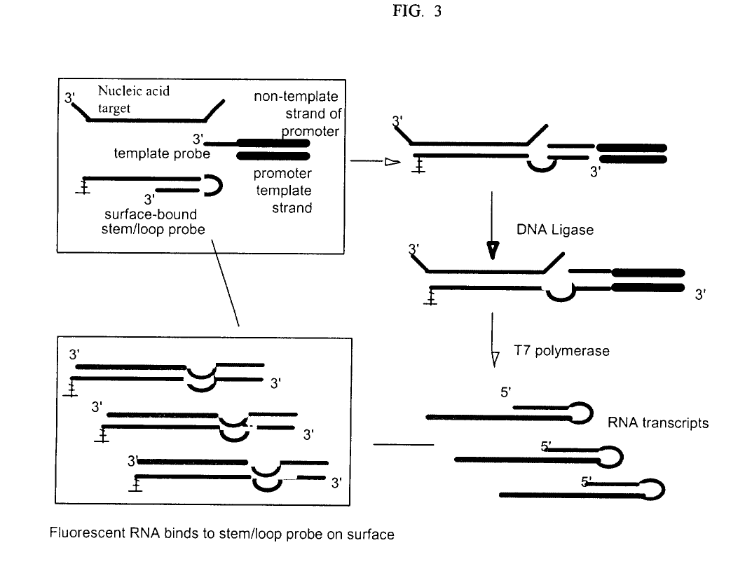

In FIG. 3 a variation of the subject method utilizes a ligase and ATP in place of the DNA polymerase. The hybridizing reagent contains the non-template strand of a promoter and a ligating reagent is included that contains the template strand of the promoter. As in the prior example, the non-template strand of the promoter is 5′ of a sequence that is complementary with the 3′ end of the short strand of the stem/loop probe. Upon displacement of the short strand by target binding to the stem/loop probe, the hybridizing reagent binds to the short strand and the ligase forms a bond between the promoter template strand and the short strand. Transcripts produced by T7 RNA polymerase may extend the full length of the stem/loop probe but will frequently be truncated because of the presence of target bound to the sequence to be transcribed. There are several advantages to this approach. The four dNTPs are not required, there is no requirement to design a probe with extra bases at the 3′ end that are not hybridized to the long strand, and excess target cannot compete with the transcripts for binding to the stem/loop probe. However when the stem/loop probe is used to bind the transcripts to a surface, the loop region of the stem/loop probe must be capable of binding its complementary sequence in the transcripts to initiate transcript-stem/loop binding. Loops of about 5 to 15 or more bases will therefore be preferred. When another means of detecting the transcripts is used it is not necessary for the loop to be a nucleic acid sequence and it may comprise any convenient linking group.

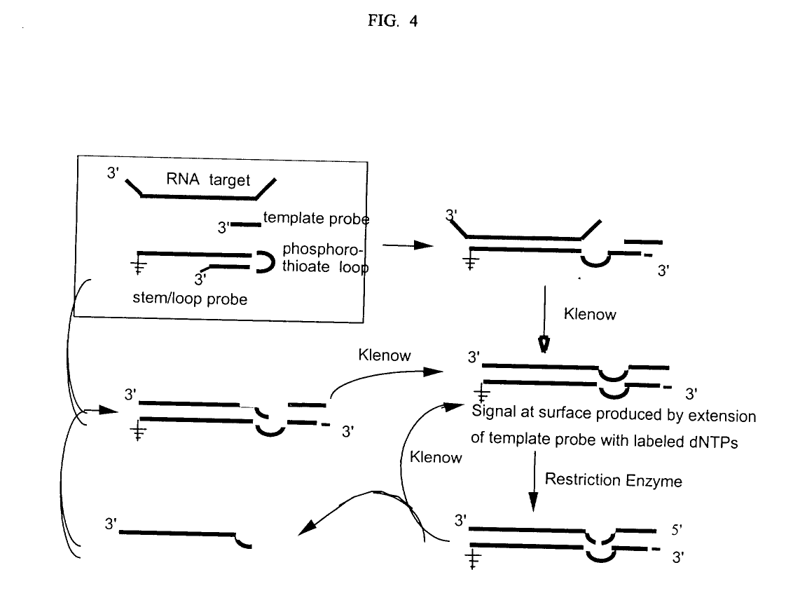

In FIG. 4, as in the prior examples the target hybridizes with the stem/loop probe causing the short strand to no longer be hybridized. In this example, the loop is comprised of a sequence that can serve as a template for DNA polymerase but is not cleaved by a restriction enzyme. For example, the loop may be a restriction site comprised of phosphorothioates (See, Eckstein, supra). Extension of the hybridizing reagent provides a double stranded structure which in this case has a restriction site. Inclusion of the appropriate restriction enzyme in the reaction mixture causes cleavage of the extended strand but not the original loop of the stem/loop probe. Subsequent extension of the 5′ end of the cut strand with the polymerase leads to displacement of the 3′ end fragment giving more of the double stranded structure. Double stranded structure is also formed by binding of the displaced 3′ end to the stem/loop probe followed by extension of the hybridizing reagent on the released short strand. The process continues to cycle providing increasing amount of the double strand. By using a labeled dNTP and having the stem/loop probe attached to a surface a fluorescent signal is generated at the surface as a function of the presence of the target.

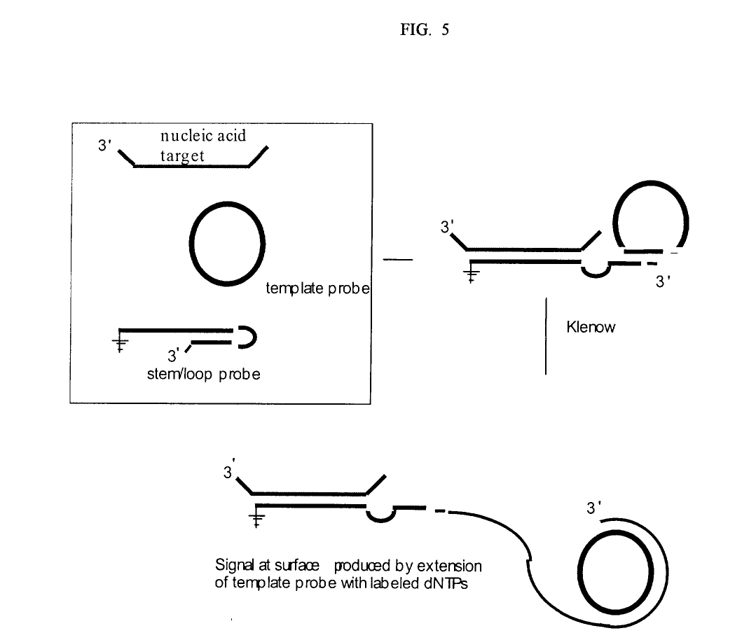

In FIG. 5, as in the prior examples the target polynucleotide binds to a stem/loop probe with the short strand at the 3′ end. Upon release of the short strand from hybridization, the short strand is available to bind to the hybridizing reagent, which in this case is circular DNA. In the presence of a DNA polymerase, the short strand is extended indefinitely with concatenated versions of the complement of the circular hybridizing reagent.

The subject method finds use in a number of applications, providing a sensitive and in many cases quantitative measure of the amount of target sequence in a sample, allowing for the independent simultaneous determination of multiple targets, up to about 100 or more, depending on the protocol and nature of the sample. The method may be used for determining an mRNA profile in as diverse situations as diagnostics for diseases, e.g. infectious diseases, cancer, genetic diseases, inflammation, autoimmune diseases, etc. The subject methods may be used in screening for drugs as to their effect on intact cells and tissue. The subject method can also be used in monitoring environmental samples, such as soils, water and air, as well as monitoring fermentation, or other industrial processes where cells are present.

For convenience, the reagents useful for the subject invention can be provided as kits, where one or more of the reagents may be combined in a single vessel in appropriate proportions. The kit would include the stem/loop probe, the hybridizing reagent and may include, the ligating reagent, DNA polymerase, RNA polymerase, ligase or restriction enzyme, along with dNTPs, NTPs and labeled binding agents and labeled nucleotide triphosphates. The kit might be for a single target or a mixture of targets, where one is interested in multiplexing the determination. For various of the assays indicated previously, one would be interested in determining the presence of a number of different nucleic acid targets, for example, where one wishes to know a transcription profile of cells.

The following examples are offered by way of illustration and not by way of limitation.

### EXAMPLE 1

### Two Step Assay of RNA Target

This example illustrates assays for a 359 nt RNA target (SEQ ID NO: 1) and an irrelevant 125 nt RNA control (SEQ ID NO: 2). A 59 nt stem/loop probe (SEQ ID NO: 3) was used that has a 39 base long strand at the 5′-end that is complementary to the target and comprised of a 24 base single stranded region, a 16 base short strand at the 3′-end that is complementary to the long strand except for a single non-complementary adenosine at the 3′ end to prevent self extension, and a 4-base loop connecting the two strands. Alternatively a 59 nt linear control probe (SEQ ID NO: 4) was used that is identical to SEQ ID NO: 3 except that only the single stranded region of the long strand is complementary to the target. The duplex region of the long strand is replaced with poly A. A common 40 nt template probe (SEQ ID NO: 5) was used with either the stem/loop or linear control probe. The template probe has a 12 base sequence at its 3′-end that is complementary with the 3′-ends of the stem/loop and control probes (SEQ ID NO: 3 and 4) and the 21 base non-template portion of a T7 promoter at its 5′-end, See the following sequence alignments: bases 67-116 of

What is claimed is:

1. A method for detecting a target nucleic acid in a sample employing as reagents a stem/loop probe having a first long strand complementary with at least a portion of said target nucleic acid, a second short strand at the other end of said probe hybridized to a portion of said long strand, and a linker joining said long and short strands, and a hybridizing reagent capable of hybridizing with said complementary sequence of said short strand to form a duplex between said stem/loop probe and said hybridizing reagent having a 3′ end capable of forming a phosphate ester bond, said method comprising:
contacting said stem/loop probe with said sampleand said hybridizing reagent, wherein said hybridizing reagent hybridizes with said short strand when said target nucleic acid is bound to said stem/loop probe;
extending said 3′-end present in said duplex; and
detecting said extension as indicating the presence of said target nucleic acid.

2. The method according to claim 1, wherein said short strand of said stem/loop probe has said 3-end and said extending comprises ligating said 3′-end to a 5′-end of said hybridizing reagent.

3. The method according to claim 1, wherein said extending comprises adding dNTPs with a DNA polymerase using at least one of the stem/loop probe and the hybridizing reagent as a template.

4. The method according to claim 1, wherein said linker comprises not more than 4 nucleotides at the junctures of the linker to said short and long strands that are complementary to each other and not complementary to the target sequence bound to said long strand.

5. A method for detecting at least one target nucleic acid in a sample employing as reagents a stem/loop probe having a long strand complementary with at least a portion of said target nucleic acid, a short strand having a sequence within 0 to 3 bases of the 3′-end of said stem/loop probe that is complementary with a portion of said long strand and a linker joining said long and short strands, and a hybridizing reagent which has a hybridizing region for complementary with the 3′-end of said short strand, said method comprising:
contacting said stem/loop probe with said sample and said hybridizing reagent and a phosphate bond forming reagent capable of linking said 3′-end to a nucleotide through said phosphate bond, wherein said linking results in attaching an active promoter to said stem/loop probe or, when said hybridizing reagent is circular, extending said 3′-end to form a concatenated DNA sequence;
when said linking results in the attaching of an active promoter, adding NTPs and RNA polymerase to produce RNA transcripts from said promoter; and
detecting the presence of said RNA transcripts or concatenated DNA sequence as indicating the presence of said target nucleic acid.

6. The method according to claim 5, wherein said phosphate-bond forming reagent is a ligase.

7. The method according to claim 5, wherein said phosphate bond-forming reagent is a DNA polymerase.

8. A method for detecting at least one target nucleic acid in a sample, said method employing as reagents a stem/loop probe having a long strand complementary with at least a portion of said target nucleic acid, a short strand having a sequence within 0-3 bases of the 3′-end of said stem/loop probe that is complementary with a portion of said long strand and a linker joining said long and short strands as a loop, and a hybridizing reagent which has a hybridizing region complementary with the 3′-end of said short strand, wherein said hybridizing reagent comprises a promoter sequence 5′ of said hybridizing region, said method comprising:
contacting said stem/loop probe with said sample and said hybridizing reagent, wherein said hybridizing reagent hybridizes with said short strand when said target nucleic acid binds to said stem/loop probe to form a complex of said stem/loop probe and said hybridizing reagent;
combining said complex with an enzyme and dNTPs for producing an extension of said stem/loop probe along said hybridizing reagent to form a promoter construct; combining said promoter construct with RNA polymerase and NTPs, whereby at least a portion of said stem/loop probe of said promoter construct is transcribed by said RNA polymerase to form RNA transcripts; and
detecting the presence of said RNA transcripts as indicating the presence of said target nucleic acid.

9. The method according to claim 8, wherein said NTPs comprise a labeled NTP.

10. The method according to claim 9, wherein said labeled NTP is a fluorescently labeled NTP.

11. The method according to claim 8, wherein said stem/loop probe is bound to a surface.

12. The method according to claim 8, wherein said hybridizing reagent comprises an identifying sequence that is transcribed and included in said RNA transcripts.

13. The method according to claim 8, wherein said DNA polymerase is a Klenow fragment and said RNA polymerase is a T bacteriophage polymerase.

14. A method for detecting at least one target nucleic acid in a sample, said method employing as reagents a stem/loop probe having a long strand complementary with at least a portion of said target nucleic acid, a short strand having a sequence within 1 to 3 bases of the 3′-end of said stem/loop probe that is complementary with a portion of said long strand wherein said 3′-end is mismatched with said long strand, and a linker joining said long and short strands as a loop, and a hybridizing reagent complementary with the 3′ -end of said short strand, wherein said hybridizing reagent comprises a promoter sequence 5′ of said hybridizable region, said method comprising:
contacting said stem/loop probe with said sample, said hybridizing reagent, DNA polymerase, and dNTPs, whereby said 31-end is extended when target nucleic acid is bound to said stem/loop probe to form a promoter construct;
combining said promoter construct, RNA polymerase and NTPs, whereby at least a portion of said stem/loop probe is transcribed to form RNA transcripts; and
detecting the presence of said RNA transcripts as indicating the presence of said target nucleic acid.

15. The method according to claim 14, wherein said NTPs comprise a labeled NTP.

16. The method according to claim 14, wherein said RNA polymerase is T bacteriophage polymerase.

17. A method for detecting at least one target nucleic acid in a sample, said method employing as reagents a stem/loop probe having a long strand complementary with at least a portion of said target nucleic acid, a short strand having a sequence within 0 to 3 bases of the 3′-end of said stem/loop probe complementary with a portion of said long strand, and a linker joining said long and short strands as a loop, and a hybridizing reagent which has a hybridizing region complementary with the 3′-end of said short strand, wherein said hybridizing reagent comprises a ligating reagent complementary to a portion of said hybridizing reagent contiguous with said hybridizing region, said method comprising:
contacting said stem/loop probe with said sample, said hybridizing reagent and a ligase; and
detecting the presence of said ligating reagent bonded to said stem/loop probe as indicating the presence of said target nucleic acid.

18. The method according to claim 17, wherein said hybridizing reagent comprises a non-template strand of a promoter sequence 5′ of said hybridizable region, said ligating reagent comprises a template strand of said promoter sequence and said detecting comprises contacting said ligating reagent bonded to said stem/loop probe with RNA polymerase and NTPs and detecting transcripts of said stem/loop probe.

19. The method according to claim 18 wherein said NTPs comprise a labeled NTP.

20. The method according to claim 18 wherein said RNA polymerase is T bacteriophage polymerase.

21. A method for detecting a target nucleic acid in a sample employing as reagents a stem/loop probe having a long strand complementary with at least a portion of said target nucleic acid, a short strand having a sequence within 0 to 3 bases of the 3′-end of said stem/loop probe that is complementary with a portion of said long strand and a linker joining said long and short strands as a loop, and a circular hybridizing reagent capable of hybridizing to at least the 3′-end of said short strand, said method comprising:
contacting said stem/loop probe with said sample, said circular hybridizing reagent, a DNA polymerase and dNTPs under conditions for extending said 3′-end to form a concatenated DNA sequence; and
detecting the presence of said concatenated DNA sequence as indicating the presence of said target nucleic acid.

22. The method according to claim 21, wherein said dNTPs comprises a labeled dNTP.

23. The method according to claim 21 wherein said DNA polymerase is the Klenow fragment.

24. A method for detecting a target nucleic acid in a sample employing as reagents a stem/loop probe having a long strand complementary with at least a portion of said target nucleic acid, a short strand having a sequence within 0 to 3 bases of the 3′-end of said stem/loop probe that is complementary with a portion of said long strand, and a linker joining said long and short strands as a loop, said linker comprising a restriction enzyme consensus sequence resistant to restriction enzyme cleavage, and a hybridizing reagent capable of hybridizing at its 3′-end to at least a portion of said short strand, said method comprising:
contacting said stem/loop probe with said sample, said hybridizing reagent, DNA polymerase, dNTPs, and a restriction enzyme specific for said consensus sequence whereby stem/loop probe bound to said target nucleic acid binds to said hybridizing reagent providing a template on which said hybridizing reagent extends by means of said DNA polymerase to form a DNA strand complementary to said stem/loop probe comprising a restriction site, cleaving said DNA strand complementary to said stem/loop probe at said restriction site with said restriction enzyme to provide a cleaved strand comprising a new DNA extension initiation site, and extending said DNA extension initiation site to provide a new DNA strand complementary to said stem/loop probe, resulting in amplification of said DNA strand complementary to said stem/loop probe; and
detecting said DNA strand complementary to said stem/loop probe as indicative of the presence of said target nucleic acid.

25. The method according to claim 24, wherein said dNTPs comprise a labeled dNTP.

26. A method for detecting at least one target nucleic acid in a sample, said method employing as reagents a stem/loop probe having a long strand complementary with at least a portion of said target nucleic acid, a short strand having a sequence starting within 0 to 3 bases of the 3′-end of said stem/loop probe that is complementary with a portion of said long strand, and a linker joining said long and short strands as a loop, and a hybridizing reagent which is complementary with the 3′-end of said short strand, said method comprising:
contacting said stem/loop probe with said sample and said hybridizing reagent wherein said stem/loop probe hybridizes with said hybridizing reagent when said target nucleic acid is present in said sample;
combining said hybridized stem/loop probe with DNA polymerase and dNTPs to cause extension of said stem/loop probe along said hybridizing reagent; and
detecting said extension as indicating the presence of said target nucleic acid.

27. The method according to claim 26, wherein said 3′-end of said short strand is mismatched with said long strand when said short strand is hybridized to said long strand.

28. The method according to claim 26, wherein said stem/loop probe is bound to a surface.

29. The method according to claim 26, wherein said linker is a polynucleotide having complementary terminal bases linking said linker to said first and second strands.

30. A kit comprising a stem/loop probe having a first strand complementary with at least a portion of a target nucleic acid, a second strand having a sequence complementary to at least a portion of said first strand, and a linker joining said first and second strands, and a hybridizing reagent hybridizable with at least a portion of said complementary sequence of said second strand to form a duplex comprising the 3′-end of at least one of said hybridizing reagent and said stem/loop probe.

31. A kit comprising a stem/loop probe having a long strand complementary with at least a portion of a target nucleic acid, a short strand having a sequence within 0 to 3 bases of the 3′-end of said stem/loop probe that is complementary with a portion of said long strand, and a linker joining said long and short strands as a loop, and a hybridizing reagent which has a hybridizing region complementary with at least a portion of said short strand.

32. The kit according to claim 31 wherein said hybridizing reagent is either circular or comprises a promoter sequence.

33. The kit according to claim 31 comprising a labeled nucleotide triphosphate.

34. The kit according to claim 31 comprising at least one of a labeled nucleotide triphosphate, DNA polymerase, or RNA polymerase.
